(19) United States
(12) Patent Application Publication
(10) Pub. No.: US 2020/0155679 A1
(43) Pub. Date: May 21, 2020

(54) ULTRASOUND RESONANCE TRIGGERING OF PAYLOAD RELEASE FROM MINIATURIZED DEVICES

(71) Applicant: BIONAUT LABS LTD., Herzliya (IL)

(72) Inventors: Michael SHPIGELMACHER, Los Angeles, CA (US); Alex KISELYOV, San Diego, CA (US)

(73) Assignee: BIONAUT LABS LTD., Herzliya (IL)

(21) Appl. No.: 16/615,327

(22) PCT Filed: May 3, 2018
(86) PCT No.: PCT/US2018/030949

§ 371 Date: Nov. 20, 2019

Related U.S. Application Data
(60) Provisional application No. 62/512,091, filed on May 29, 2017.

(51) Int. Cl. A61K 41/00; A61M 37/00; A61K 9/00

(52) U.S. Cl. CPC A61K 41/0028; A61K 9/0009; A61K 9/0024; A61M 37/0092

(57) ABSTRACT

A carrier device and methods of use, for implanting in biological tissue and releasing a medical payload in the biological tissue according to remote ultrasound trigger pulses. The carrier device includes at least one internal resonant element with a resonance frequency in the ultrasound range. When an ultrasound pulse at the resonance frequency is sent through the tissue, the resonant element vibrates at high amplitude and raises the internal pressure of the carrier and releases the payload. In some embodiments, payload release can be started, stopped, and restarted at a later time or place. Individual carrier devices can be selectively triggered by providing different resonance frequencies, and external resonant elements can provide propulsion and navigation capabilities.

### CROSS REFERENCE TO RELATED APPLICATIONS

This application claims benefit of U.S. Provisional Patent Application Ser. No. 62/512,091, filed May 29, 2017, the priority date of which is hereby claimed.

### BACKGROUND

Ultrasound (“US”)-based methods currently exist for remotely triggering release of a medical payload, such as drugs and diagnostic aids, from particles or devices implanted in a living tissue. Remotely-triggered payload release is desirable in supporting specific clinical goals, including, but not limited to examples such as:

release of medical payload only when the carrier is in a specific location (e.g., a tumor);

release of medical payload only at a specific time (e.g., at a particular step of a clinical procedure); or

release of medical payload only at particular concentration and/or quantity as determined by a clinical protocol.

Existing US triggering methods rely on a variety of effects, including:

thermal/mechanical effects based on US-induced cavitation, which causes localized heating due to vibration; and increased speed of diffusion and/or changes in localized medium properties which increase diffusion;

mechanical degrading/rupturing of carrier leading to payload release;

shape change of the carrier; and
changes to characteristics of the surrounding biological tissue into which the payload is being released (e.g., sonoporation), resulting in improved payload diffusion/absorption through the tissue.

Unfortunately, current methods suffer from a number of serious drawbacks. First, none of the current methods supports more than a subset of the following clinical requirements:

The ability to penetrate more than about 10 cm of tissue penetration depth (10 cm or greater, a limit for diagnostic US at frequencies of 7 MHz and up); and the ability to customize the depth of tissue at which release occurs.

Support for customizable US frequency ranges (KHz-MHz), for compatibility with existing medical US equipment, and to minimize invasiveness in tissue. For example, cavitation-based methods are typically most effective in the KHz range using High-Intensity Focused Ultrasound (HIFU), while polymer degradation methods are more effective in the KHz range and polymer conformation change methods are practical in the MHz range (diagnostic US).

Support for gradual payload release over a controllable time period; alternatively, support for an on-off switchable release functionality (rather than a single release pulse). In contrast, methods relying on degradation of a uniform polymer encasing the payload are by design irreversible and lack a gradual release functionality. Similarly, none of the described methods reliably support repetitive stop-treat-go cycles, namely gradual regimented payload release in the pre-set location.

Second, none of the existing methods supports individual control of multiple payload carriers in the same tissue region (i.e., releasing payload selectively from only specific carriers out of many carriers located within the same region exposed to the US trigger).

It would therefore be desirable to have implantable devices and methods thereof, which overcome the above restrictions of the current capabilities. This goal is attained by embodiments of the present invention.

### SUMMARY

According to various embodiments of the present invention, there is provided an implantable payload carrier device with at least one resonant element having a predetermined resonance frequency at an ultrasound frequency, to implement:

customizable tissue penetration depths;

support for customizable US frequency ranges;

gradual and on/off switchable and regimented payload release capabilities;

individual control of multiple payload carriers in the same tissue region; and

methods for use of the above devices.

Certain embodiments of the present invention rely on ultrasound (“US”) for remote triggering and navigation of carriers implanted in living tissue. Other embodiments combine ultrasound with other external physical stimuli, non-limiting examples of which include: electromagnetic fields, phenomena, and effects; and thermodynamic phenomena and effects, including both temperature and pressure effects.

The terms “carrier device” and “carrier” herein denote any object that is implantable in biological tissue, and is capable of carrying and releasing a medical payload into the tissue. The term “medical payload”, or equivalently the term “payload” used in a medical context is understood herein to include any substance or material, a combination of several relevant therapeutic materials, diagnostics or a combination of therapeutic and diagnostics. In certain embodiments of the present invention, a fluid payload is used; the term “fluid” herein denotes that the payload readily yields to pressure and is capable of flowing. In certain embodiments of the present invention, a solid payload is used; the term “solid” herein denotes that the payload yields to internal or external stimuli and can be released in the form of discrete particles. The term “device” (with reference to a carrier) herein denotes a carrier which is fabricated by known manufacturing techniques, including, but not limited to, 3D printing, molding, casting, etching, lithography, thin-film technologies, deposition technologies, and the like. The term “particle” (with reference to a carrier) herein denotes a carrier of up to macromolecular scale.

In various embodiments of the present invention, carrier devices are miniaturized for implantation in biological tissues. The term “miniaturized” (with reference to a carrier) herein denotes a carrier of small size, including, but not limited to: carriers of millimeter to centimeter scale; carriers of micrometer (“micron”) scale, referred to as “carrier micro-devices”; carriers of nanometer scale (including hundreds of nanometers), referred to as “carrier nano-devices”; and carriers of macromolecular scale, referred to as “carrier particles”. Not only are the carriers themselves of the size scales as indicated above, but the carriers' individual components are also of comparable scale.

In one embodiment, this invention provides a carrier device for implanting in a region of biological tissue to release a medical payload in the tissue, the carrier device comprising:

a cavity for containing the medical payload, wherein the cavity has an internal pressure;

a resonant element having a predetermined resonance frequency, and arranged so that the resonant element, when resonating at the predetermined resonance frequency, increases the internal pressure of the cavity and causes the medical payload to be released from the cavity into the tissue;

wherein the predetermined resonance frequency is an ultrasound frequency.

In one embodiment, the cavity is sealed by a flexible seal, which opens when the internal pressure exceeds a predetermined threshold value. In one embodiment, the flexible seal closes when the internal pressure is below the predetermined threshold value. In one embodiment, the cavity has a small hole through which the medical payload diffuses when the internal pressure exceeds a predetermined threshold value. In one embodiment, the medical payload stops diffusing when the internal pressure falls below the predetermined threshold value. In one embodiment, the resonant element is the cavity. In one embodiment, the resonant element is a flexible cantilever inside the cavity. In one embodiment, the resonant element is a membrane inside the cavity.

In one embodiment, the device further comprising at least one flexible cantilever attached to the outside of the carrier device, wherein the at least one flexible cantilever has a predetermined propulsion resonance frequency, and arranged so that the external flexible cantilever, when resonating at the predetermined propulsion resonance frequency, propels the carrier device through the biological tissue, and wherein the predetermined propulsion resonance frequency is an ultrasound frequency.

In one embodiment, this invention provides a method for releasing a medical payload in biological tissue, the method comprising:

selecting a carrier device containing the medical payload for implant in the biological tissue, wherein the carrier device includes a resonant element for releasing the medical payload, wherein the resonant element has a predetermined release resonance frequency, and wherein the predetermined release resonance frequency is an ultrasound frequency;

implanting the carrier device in the biological tissue; and

pulsing of ultrasound at the predetermined release resonance frequency, to release the medical payload in the biological tissue.

In one embodiment, the method further comprising: repeating the pulsing of ultrasound at the predetermined release resonance frequency, to repeat releasing the medical payload in the biological tissue.

In one embodiment, the carrier device further includes a propulsion resonant element for propelling the carrier device, wherein the propulsion resonant element has a predetermined propulsion resonance frequency, and wherein the predetermined propulsion resonance frequency is an ultrasound frequency, and where the predetermined propulsion resonance frequency is not the same as the predetermined release resonance frequency, the method further comprising: pulsing of ultrasound at the predetermined propulsion resonance frequency, to propel the carrier device through the biological tissue.

### BRIEF DESCRIPTION OF THE DRAWINGS

The subject matter disclosed may best be understood by reference to the following detailed description when read with the accompanying drawings in which:

FIG. 1A conceptually illustrates a cross-section of a carrier device with releasable payload containment according to an embodiment of the present invention.

FIG. 1B conceptually illustrates a cross-section of a carrier device with releasable payload containment according to another embodiment of the present invention.

FIG. 2A conceptually illustrates a cross-section of a carrier device having a cantilever expeller component according to an embodiment of the present invention.

FIG. 2B conceptually illustrates payload release by the carrier device of FIG. 2A.

FIG. 3A conceptually illustrates a cross-section of a carrier device having a cantilever expeller component according to another embodiment of the present invention.

FIG. 3B conceptually illustrates payload release by the carrier device of FIG. 3A.

FIG. 4A conceptually illustrates a cross-section of a carrier device having a membrane expeller component according to an embodiment of the present invention.

FIG. 4B conceptually illustrates payload release by the carrier device of FIG. 4A.

FIG. 5 conceptually illustrates a cross-section of a carrier device according to an embodiment of the present invention, which provides a cantilever expeller component and cantilever propelling components.

FIG. 6 is a flowchart of a method according to an embodiment of the present invention.

For simplicity and clarity of illustration, elements shown in the figures are not necessarily drawn to scale, and the dimensions of some elements may be exaggerated relative to other elements. In addition, reference numerals may be repeated among the figures to indicate corresponding or analogous elements.

### DETAILED DESCRIPTION

Various embodiments of the present invention provide a carrier device containing a resonant element which actuates release of the carrier's payload. The term “resonant element” herein denotes any component or part of the carrier device which vibrates in response to physical stimuli and has a specific predetermined resonant frequency. Vibrational energy from a physical stimulus at or near the resonant frequency accumulates in the resonant element, which causes the amplitude of the vibrations to increase significantly at the resonant frequency. Physical stimulus that is not at or near the resonance frequency, however, does not accumulate in the resonant element, and thus the resonant element's response at or near the resonant frequency is an important characteristic of the resonant element. According to certain embodiments of the present invention, the resonance frequency of a resonant element of a carrier is referred to as the resonance frequency of the carrier itself.

According to these embodiments, the increased amplitude of vibration of the resonant element cause an increase in pressure of the payload, particularly in the case of a fluid payload, and the increased pressure in turn causes release of the payload from the carrier. Furthermore, according to these embodiments, the resonant frequency of the resonant element is predetermined to be in the range of ultrasound, so that the remote triggering of a particular carrier device to release the payload is accomplished by sending pulses of ultrasound tuned to the resonant frequency of the carrier device's resonant element.

In some embodiments of the present invention, a carrier device and its component parts are miniaturized. According to some embodiments, the diameter or actual length of the overall device is selected from: between 100 and 5,000 micrometers, between 10 and 100 micrometers, between 1 and 10 micrometers, between 200 and 1,000 nanometers, and any combination thereof. According to some embodiments, the diameter or actual length of the overall device is from 200 nanometers up to 5,000 micrometers.

In some embodiments of the present invention, a carrier device comprises a shape selected from elongated, axisymmetric, centrosymmetric, chiral, random and a combination thereof. In some embodiments of the present invention, a resonant element comprises a configuration selected from an elongated shape, a film, a wire, a string, a strip, a sheet, a plug, a membrane, flagellum, coil, helix, arm, joint and a combination thereof.

Following are detailed descriptions of some non-limiting embodiments, with reference to drawings thereof.

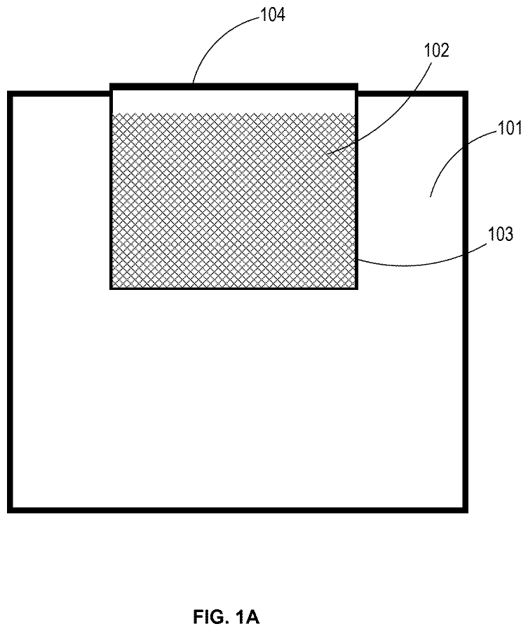

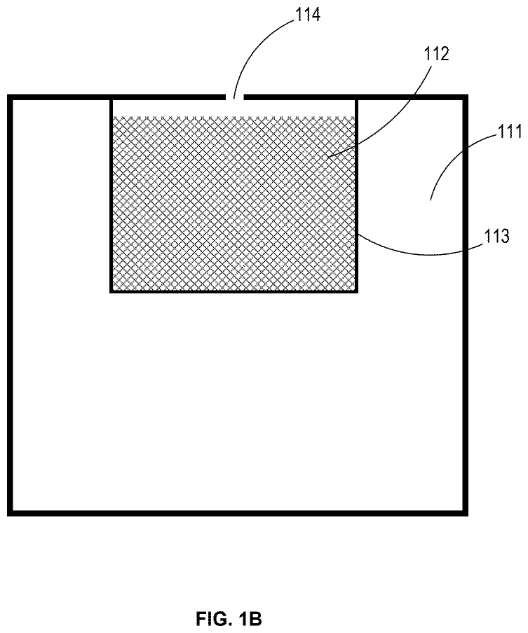

FIG. 1A conceptually illustrates a cross-section of a carrier device 101 with a releasable fluid payload 102 contained in a cavity 103, according to an embodiment of the present invention. Cavity 103 has an internal pressure, which may vary according to certain conditions, including, but not limited to: temperature; and external pressure. The term “internal pressure” herein denotes a pressure inside the cavity relative to a pressure immediately outside the cavity. The internal pressure in cavity 103 also applies to the contents of cavity 103. That is, whatever the internal pressure in cavity 103 may be, that same internal pressure is applied to whatever is inside cavity 103. Likewise, whatever internal pressure is applied to the contents of cavity 103 is also the internal pressure of cavity 103 itself. A flexible membrane 104 covers an opening in cavity 103. Carrier device 101 is implanted or placed within biological tissue. In one embodiment, cavity 103 is a resonant element. Flexible membrane 104 normally seals payload 102 from release into the biological tissue, but opens to the outside when the internal pressure of cavity 103 exceeds a predetermined threshold value. In a related embodiment, flexible membrane 104 is polymer-based. According to some embodiments, the volume of the cavity is selected from between 5% and 95% of the carrier device. According to some embodiments, the volume of the cavity is selected from 5%, 10%, 15%, 20%, 25%, 30%, 35%, 40%, 45%, 50%, 55%, 60%, 65%, 70%, 75%, 80%, 85%, 90% or 95% of the volume of the carrier device.

FIG. 1B conceptually illustrates a cross-section of a carrier device 111 with a releasable fluid payload 112 contained in a cavity 113, according to an embodiment of the present invention. A small hole 114 (whose dimensions are small relative to cavity 113) allows payload 112 to exit cavity 113, but under normal conditions (at normal body temperature, in the absence of external ultrasound stimulation), diffusion of payload 112 out of cavity 113 via hole 114 is negligible. In one embodiment, cavity 103 itself is a resonant element.

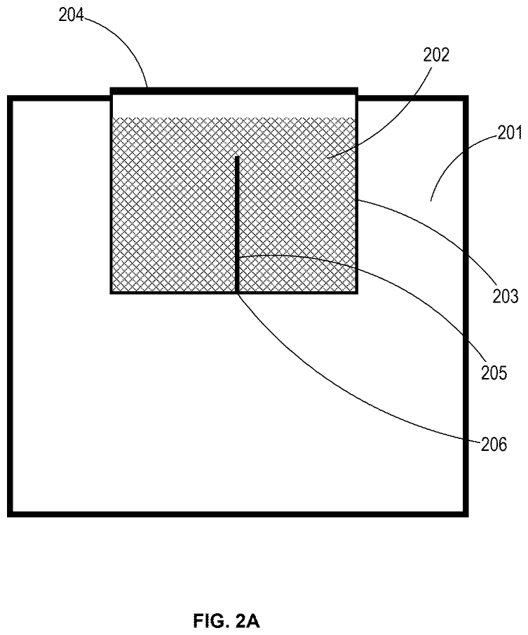

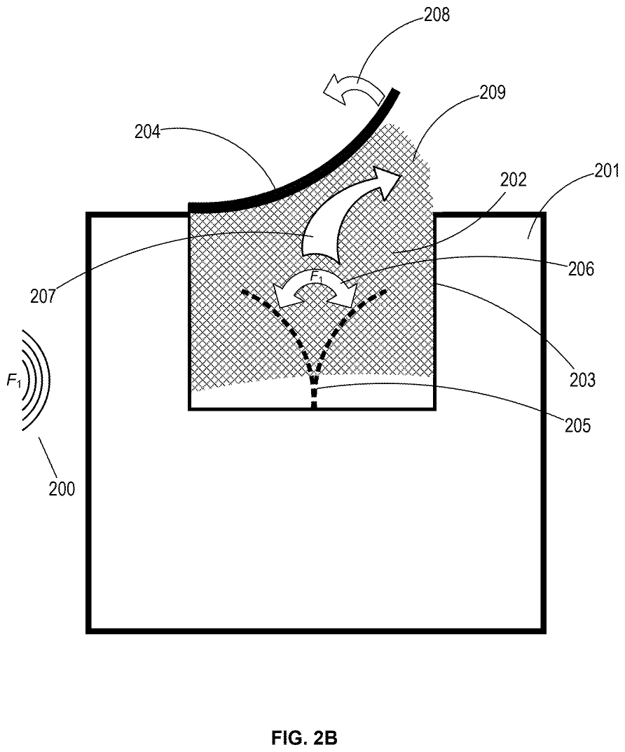

FIG. 2A conceptually illustrates a cross-section of a carrier device 201 with releasable fluid payload 202 contained in a cavity 203 with a flexible membrane seal 204 which normally seals payload 202 from release into the biological tissue, but opens to the outside when the internal pressure of cavity 203 exceeds a threshold value. Inside cavity 203 is located a cantilever expeller component 205 according to an embodiment of the present invention. The term “cantilever” herein denotes an elongated element that is attached only at one end, in this case an end 206 affixed to an inside wall of cavity 203. Furthermore, a “cantilever” according to embodiments of the present invention is a flexible and/or deformable vane-like element which, when immersed in a fluid payload and when flexed and/or deformed increases the pressure on the fluid payload and causes the fluid payload to flow or change direction of flow. In a related embodiment, multiple cantilever expeller components are used.

FIG. 2B conceptually illustrates the release of fluid payload 202 by carrier device 201, according to the illustrated embodiment of the invention. An external ultrasound pulse 200 of frequency F₁ is introduced into the environment of carrier device 201. Cantilever expeller 205 has been fabricated by appropriate choices of geometry and material to have a predetermined resonant frequency of F₁, and therefore begins to resonate under the influence of external ultrasound pulse 200, vibrating at high amplitude at resonant frequency F₁ as shown by double-headed arrow 206, thereby increasing the internal pressure within fluid payload 202 in a direction 207. When the increased internal pressure exceeds a predetermined threshold value, it causes flexible membrane seal 204 to open in a direction 208, thereby uncovering the opening of cavity 203, and in turn leading to a release 209 of fluid payload 202. Thus, external ultrasound pulse 200 of frequency F₁ serves as a trigger for release of fluid payload 202. According to a related embodiment of the invention, flexible membrane seal 204 opens reversibly, and when external ultrasound pulse 200 is stopped, cantilever expeller 205 also stops vibrating, and the internal pressure drops below the threshold, so that flexible membrane seal 204 returns to its initial closed position (as shown in FIG. 2A) and fluid payload release 209 stops. This feature allows on-off operation of carrier 201.

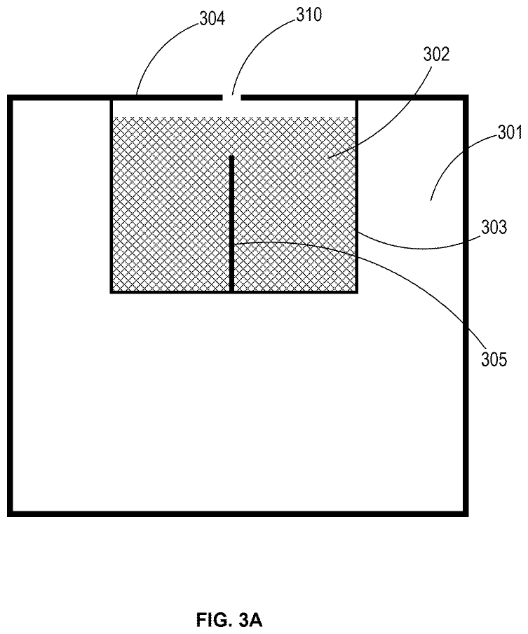

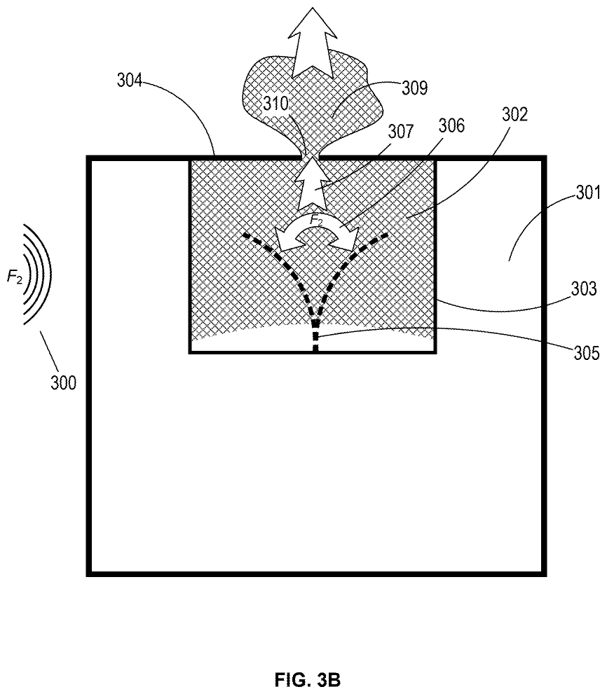

FIG. 3A conceptually illustrates a cross-section of a carrier device 301 with a releasable fluid payload 302 contained in a cavity 303, according to an embodiment of the present invention. Inside cavity 303 is located a cantilever expeller component 305 according to an embodiment of the present invention. In a related embodiment, multiple cantilever expeller components are used. An external wall 304 of cavity 303 has a small hole 310 (whose dimensions are small relative to cavity 303) allows payload 302 to exit cavity 303, but under normal conditions (at normal body temperature, in the absence of external ultrasound stimulation), diffusion of payload 302 out of cavity 303 via hole 304 is negligible. In a related embodiment, payload 302 is released through hole 310 when the internal pressure of cavity 303 exceeds a predetermined threshold value, and stops being released when the internal pressure of cavity 303 falls below the threshold value.

FIG. 3B conceptually illustrates the release of fluid payload 302 by carrier device 301, according to the illustrated embodiment of the invention. An external ultrasound pulse 300 of frequency F₂ is introduced into the environment of carrier device 301. Cantilever expeller 305 has been fabricated by appropriate choices of geometry and material to have a predetermined resonant frequency of F₂, and therefore begins to resonate under the influence of external ultrasound pulse 300, vibrating at high amplitude at resonant frequency F₂ as shown by double-headed arrow 306, thereby increasing the internal pressure within fluid payload 302 in a direction 307. The increased internal pressure increases the pressure of fluid payload 302, thereby increasing the diffusion of fluid payload 302 through hole 310, leading to a release 309 of fluid payload 302. Thus, external ultrasound pulse 300 of frequency F₂ serves as a trigger for release of fluid payload 302. According to a related embodiment, when external ultrasound pulse 300 is stopped, cantilever expeller 305 also stops vibrating, so that the internal pressure drops and the diffusion of fluid payload 309 through hole 310 returns to its normal negligible level (FIG. 3A) and fluid payload release 309 stops. This feature allows on-off operation of carrier 301.

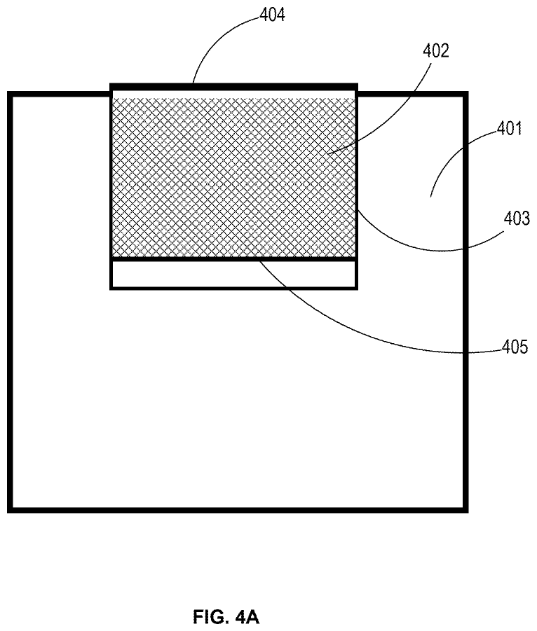

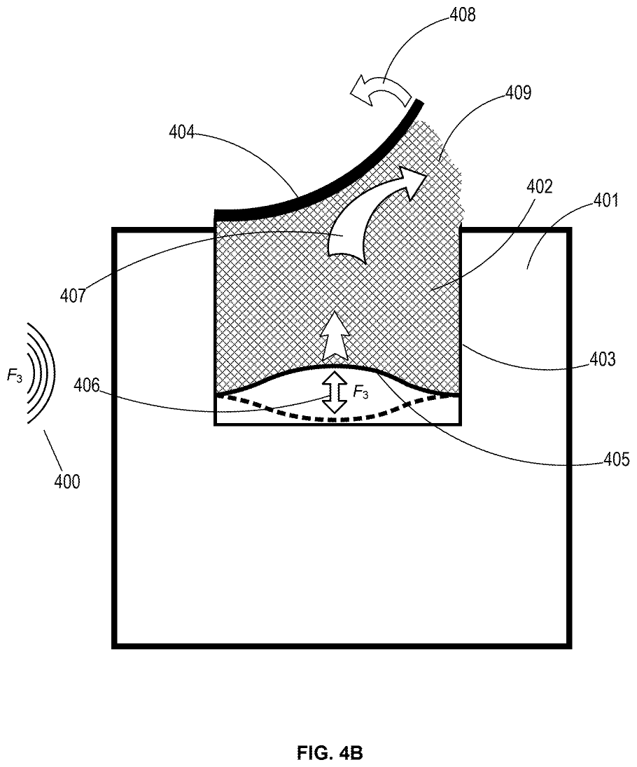

FIG. 4A conceptually illustrates a cross-section of a carrier device 401 with releasable fluid payload 402 contained in a cavity 403 with a flexible membrane seal 404 which normally seals payload 402 from release into the biological tissue, but opens to the outside when the internal pressure of cavity 403 exceeds a threshold value. Inside cavity 403 is located a membrane expeller component 405 according to an embodiment of the present invention.

FIG. 4B conceptually illustrates the release of fluid payload 402 by carrier device 401, according to the illustrated embodiment of the invention. An external ultrasound pulse 400 of frequency F₃ is introduced into the environment of carrier device 401. Membrane expeller 405 has been fabricated by appropriate choices of geometry and material to have a predetermined resonant frequency of F₃, and therefore begins to resonate under the influence of external ultrasound pulse 400, vibrating at high amplitude at resonant frequency F₃ as shown by double-headed arrow 406, thereby increasing the internal pressure within fluid payload 402 in a direction 407. The increased pressure causes flexible membrane seal 404 to open in a direction 408, thereby uncovering the opening of cavity 403, and in turn leading to a release 409 of fluid payload 402. Thus, external ultrasound pulse 400 of frequency F₃ serves as a trigger for release of fluid payload 402. According to a related embodiment of the invention, flexible membrane seal 404 opens reversibly, and when external ultrasound pulse 400 is stopped, membrane expeller 405 also stops vibrating, so that the internal pressure drops and flexible membrane seal 404 returns to its initial closed position (as shown in FIG. 4A) and fluid payload release 409 stops. This feature allows on-off operation of carrier 401.

The embodiments shown in the above-referenced drawings and descriptions are non-limiting; other ultrasound-sensitive configurations are also possible in keeping with the present invention. In particular, one or more flexible mechanical components of different shapes and materials may be located at different positions inside the cavity relying on the principles describe above to achieve the effect of payload release.

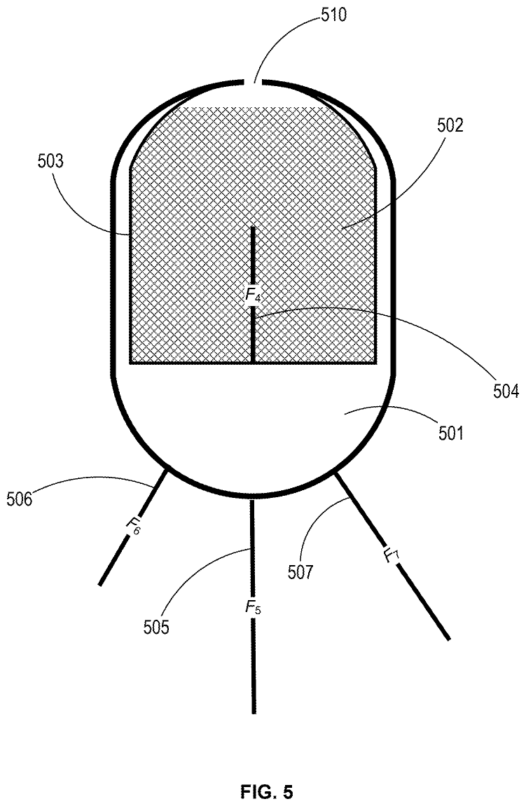

FIG. 5 conceptually illustrates a cross-section of a carrier device 501 according to an embodiment of the present invention, which provides a cantilever release component 504 and cantilever propelling components 505, 506, and 507. Carrier device 501 features a cavity 503 containing a fluid payload 502, and a small hole 510 for operation similar to carrier device 301 illustrated in FIG. 3A and FIG. 3B. Cantilever propulsion components 505, 506, and 507 are attached to the outside of carrier device 501 and provide propelling force and directional navigation for carrier device 501 in a fluid environment, or an environment characterized by having localized fluid dynamic properties. When cantilever propulsion elements 505, 506, and/or 507 resonate at a predetermined propulsion resonance frequency, they cause carrier device 501 to be propelled through the biological tissue in which it has been implanted. In a related embodiment, each cantilever has a different resonant frequency, as illustrated in FIG. 5, where cantilever release component 504 has a predetermined release resonance frequency F₄, while cantilever propulsion components 505, 506, and 507 have predetermined propulsion resonance frequencies F₅, F₆, and F₇, respectively, thereby allowing each cantilever component to be activated individually by sending pulses of external ultrasound at the corresponding frequency. Activating individual propelling components 505, 506, and 507 separately or in specific combinations allows directional control of the propulsion.

According to embodiments of the present invention, the flexible mechanical components can be made of a variety of flexible materials, such as the polymer PET. Representative methods of fabrication include but are not limited to: template-assisted synthesis, as exemplified by direct or vertical laser writing; photolithographic etching and spinning techniques.

As a non-limiting example of a PET cantilever, choose the length to be 90 microns, the width and thickness to be 30 microns. With a Young modulus of 2×10⁹/m² and a density of 1.4 g/cm², the resonant frequency is approximately 200 KHz (utilizing standard formulas for cantilever mechanical resonance orthogonal to cantilever length dimension). Appropriate adjustments of the geometrical parameters and material choice allow changing the resonant frequency by a factor of 100 or more, either up or down, easily covering the range of KHz to MHz as needed, which covers the frequency range of typical ultrasound pulses.

The selected resonance frequency F determines the possible penetration depth, and also enables the individual control of several carriers in a single region. As noted in the figures and in the corresponding descriptions above, each carrier can have a different resonant frequency, thus allowing individual activation of a single carrier by US pulses at specific frequencies.

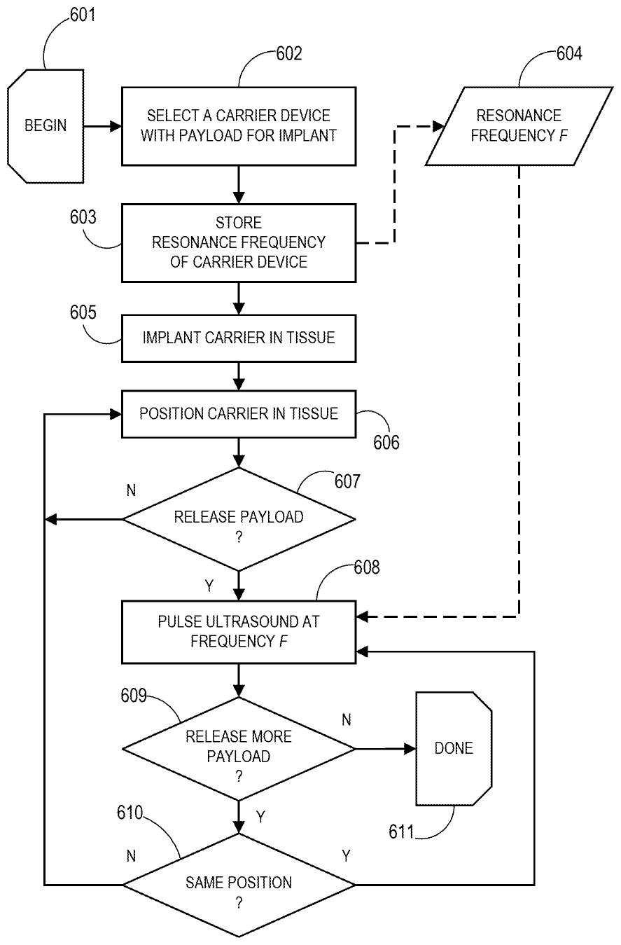

FIG. 6 is a flowchart of a method according to an embodiment of the present invention. The method begins at a point 601 and then in a step 602, a carrier device with a desired payload is selected for implant into a region of tissue (where the carrier is as provided by other embodiments of the present invention). Before implant, in a step 603 the resonance frequency of the carrier device F is stored as data 604. Next, in a step 605 the carrier is implanted in the tissue, and in a step 606, the implanted carrier is positioned in the tissue as desired. At a decision point 607 it is determined if the carrier is positioned properly, and if it is the proper time to release the payload. If not, step 606 is repeated until the desired conditions for payload release are met, in which case a step 608 is performed. In step 608 an ultrasound signal of frequency F is pulsed. As previously described, this will cause the carrier to release payload into the tissue. As provided by other embodiments, when the pulse is completed, the carrier will stop releasing payload. Thus, at a decision point 609 it is determined if more payload should be released. If more payload is to be released, at a decision point 610 it is determined whether additional payload should be released at the same location in the tissue. If so, then step 608 is repeated directly. If not, however, then step 606 is repeated to reposition the carrier. If no additional payload should be released, then decision point 609 completes the method by ending the procedure at a point 611.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A carrier device for implanting in a region of biological tissue to release a medical payload in the tissue, the carrier device comprising:
a cavity for containing the medical payload, wherein the cavity has an internal pressure;
a resonant element having a predetermined resonance frequency, and arranged so that the resonant element, when resonating at the predetermined resonance frequency, increases the internal pressure of the cavity and causes the medical payload to be released from the cavity into the tissue;
wherein the predetermined resonance frequency is an ultrasound frequency.

2. The carrier device of claim 1, wherein the cavity is sealed by a flexible seal, which opens when the internal pressure exceeds a predetermined threshold value.

3. The carrier device of claim 2, wherein the flexible seal closes when the internal pressure is below the predetermined threshold value.

4. The carrier device of claim 1, wherein the cavity has a small hole through which the medical payload diffuses when the internal pressure exceeds a predetermined threshold value.

5. The carrier device of claim 4, wherein the medical payload stops diffusing when the internal pressure falls below the predetermined threshold value.

6. The carrier device of claim 1, wherein the resonant element is the cavity.

7. The carrier device of claim 1, wherein the resonant element is a flexible cantilever inside the cavity.

8. The carrier device of claim 1, wherein the resonant element is a membrane inside the cavity.

9. The carrier device of claim 1, further comprising at least one propulsion resonant element attached to the outside of the carrier device for propelling the carrier device, wherein the propulsion resonant element has a predetermined propulsion resonance frequency, and arranged so that the propulsion resonant element, when resonating at the predetermined propulsion resonance frequency, propels the carrier device through the biological tissue, and wherein the predetermined propulsion resonance frequency is an ultrasound frequency, and where the predetermined propulsion resonance frequency is not the same as the predetermined resonance frequency.

10. The carrier device of claim 1, wherein the propulsion resonant element comprises a flexible cantilever.

11. A method for releasing a medical payload in biological tissue, the method comprising:
selecting a carrier device containing the medical payload for implant in the biological tissue, wherein the carrier device includes a resonant element for releasing the medical payload, wherein the resonant element has a predetermined release resonance frequency, and wherein the predetermined release resonance frequency is an ultrasound frequency;
implanting the carrier device in the biological tissue; and
pulsing of ultrasound at the predetermined release resonance frequency, to release the medical payload in the biological tissue.

12. The method of claim 11, further comprising:
repeating the pulsing of ultrasound at the predetermined release resonance frequency, to repeat releasing the medical payload in the biological tissue.

13. The method of claim 11, wherein the carrier device further includes a propulsion resonant element for propelling the carrier device, wherein the propulsion resonant element has a predetermined propulsion resonance frequency, and wherein the predetermined propulsion resonance frequency is an ultrasound frequency, and where the predetermined propulsion resonance frequency is not the same as the predetermined release resonance frequency, the method further comprising:
pulsing of ultrasound at the predetermined propulsion resonance frequency, to propel the carrier device through the biological tissue.

14. The method of claim 13, wherein the propulsion resonant element comprises a flexible cantilever.
